(19) United States
(12) Patent Application Publication
(10) Pub. No.: US 2004/0266832 A1
(43) Pub. Date: Dec. 30, 2004

(54) Crystal forms of 2-(3-difluoromethyl-5-phenyl-pyrazol-1-yl)-5-methanesulfonyl pyridine

(76) Inventors: Zheng J. Li, Quaker Hill, CT (US); Robert J. Rafka, Stonington, CT (US); David H. B. Ripin, Old Saybrook, CT (US); Michael J. Castaldi, Pawcatuck, CT (US)

Correspondence Address: PHARMACIA & UPJOHN, 301 HENRIETTA ST, 0228-32-LAW, KALAMAZOO, MI, 49007, US

(21) Appl. No.: 10/875,705

(22) Filed: Jun. 24, 2004

Related U.S. Application Data
(60) Provisional application No. 60/482,679, filed on Jun. 26, 2003.

(51) Int. Cl. A61K 31/4439; C 07D 4 3/02

(52) U.S. Cl. 514/341; 546/275.4

(57) ABSTRACT

The invention concerns crystalline forms of 2-(3-difluromethyl-5-phenyl-pyrazol-1-yl)-5-methanesulfonyl-pyridine, their preparation and use in pharmaceuticals.

### CROSS REFERENCE TO RELATED APPLICATIONS

This application claims the benefit of U.S. provisional application Ser. No. 60/482,679 filed on 26 Jun. 2003, under 35 USC 119(e)(i), which is incorporated herein by reference in its entirety.

### BACKGROUND OF THE INVENTION

1. Field of the Invention

The invention pertains to 2-(3-difluromethyl-5-phenyl-pyrazol-1-yl)-5-methanesulfonyl-pyridine in its crystal forms, in particular its polymorphic forms, and to methods for selective production of same, pharmaceutical compositions comprising said crystal forms and methods of treating pain and/or inflammation using said crystal forms.

2. Description of the Prior Art

The properties manifested by a given compound are a function of chemical composition and structure. Among the latter is crystalline form. When a compound exists in two or more crystalline forms it is said to be polymorphic. Polymorphism often arises as a result of the particular processing conditions used to synthesize the compound. Typically, one crystalline form is more stable than the other(s). This is sometimes manifested by the less stable form (the metastable form) having a melting point lower than the melting point of the stabler form. Moreover, it is not uncommon for one crystalline form to be convertible to another, either over time or under particular circumstances.

In the pharmaceutical field, the polymorphic state of a compound can have a profound influence on the bioavailability and storage properties of a drug comprised of the compound. For example, one polymorph may be more readily dissolvable than another, or may be more ingestible, or may have a longer shelf life, or may exhibit controlled release behavior that is manipulable under certain conditions. Given these factors, it is important to characterize polymorphism as may occur in pharmaceutical compounds.

The compound 2-(3-difluromethyl-5-phenyl-pyrazol-1-yl)-5-methanesulfonyl-pyridine (C₁₆H₁₃F₂N₃O₂S; Molecular Weight=349.36) has the structure shown below in Formula I:

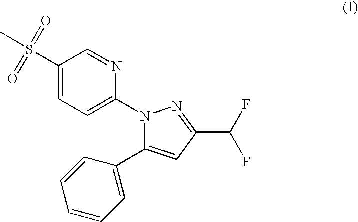

The compound of Formula I is a cyclo-oxygenase-2 (COX-2) inhibitor useful in treating acute and chronic pain, particularly in mammals including humans, livestock and companion animals, such as dogs and cats. Among the treatments contemplated by the compound of Formula I are those for the alleviation of inflammation, e.g. associated with disorders such as arthritis, neurodegeneration, and colon cancer. For example, U.S. patent application Ser. No. 09/724446, filed Nov. 28, 2000, the entire contents of which are incorporated herein by reference, at Example 80 demonstrates the utility of the compound of Formula I as a COX-2 inhibitor in the treatment of mammalian pain and inflammation.

COX-2 is an inducible enzyme whose presence is believed associated with prostaglandins, which are lipid compounds. Some prostaglandins are theorized to be mediators of inflammation, the production of which is thought to ensue from cell perturbation, such as caused by injury or disease. Inhibition of the COX-2 isozyme is therefore advantageous inasmuch as reduction of related inflammation and pain occurs. Polymorphism, as may obtain in medicaments such as COX-2 inhibitors like 2-(3-difluromethyl-5-phenyl-pyrazol-1-yl)-5-methanesulfonyl-pyridine, is a property desirably established inasmuch as it can effect stability, uptake and the like, as before stated.

### SUMMARY OF THE INVENTION

The present invention is premised on the polymorphism aforesaid as pertains to the compound of Formula I. Specifically, the invention relates to a crystalline form of 2-(3-difluromethyl-5-phenyl-pyrazol-1-yl)-5-methanesulfonyl-pyridine; the preferable crystalline forms of same being selected from the group consisting of:

(a) Form A characterized by a powder X-ray diffraction pattern having peaks at 2-θ values of approximately 11.4, 12.8, 17.4, 18.1, 18.5, 19.8, 20.7, 21.6, 23.0, 25.5 and 27.1; and

(b) Form B characterized by a powder X-ray diffraction pattern having peaks at 2-θ values of approximately 6.1, 8.6, 12.5, 17.2, 17.7, 18.5, 20.9, 22.8, 23.4, 24.7 and 27.1.

The invention also concerns a method of making said crystalline Form A or crystalline Form B.

In another practice, the invention is directed to a pharmaceutical composition comprising a crystalline form of said pyridine, e.g. one of crystalline Forms A or B, e.g. in a therapeutically effective amount.

The invention still further relates to a method of treating inflammation and/or pain by administering to a subject in need of such treatment a therapeutically effective amount of crystalline form of said pyridine, e.g. crystalline Form A or Form B.

### BRIEF DESCRIPTION OF THE DRAWINGS

FIG. 1A is a powder X-ray diffraction pattern of the crystalline polymorph Form A of 2-(3-difluromethyl-5-phenyl-pyrazol-1-yl)-5-methanesulfonyl-pyridine; FIG. 1B is a calculation of powder X-ray diffractive pattern from single crystal data for Form A.

FIG. 2 is a powder X-ray diffraction pattern of the crystalline polymorph Form B of 2-(3-difluromethyl-5-phenyl-pyrazol-1-yl)-5-methanesulfonyl-pyridine.

### DETAILED DESCRIPTION OF THE INVENTION

In a preferred practice, the compound 2-(3-difluromethyl-5-phenyl-pyrazol-1-yl)-5-methanesulfonyl-pyridine shown in Formula I exists as two polymorphs denoted herein as Form A and Form B.

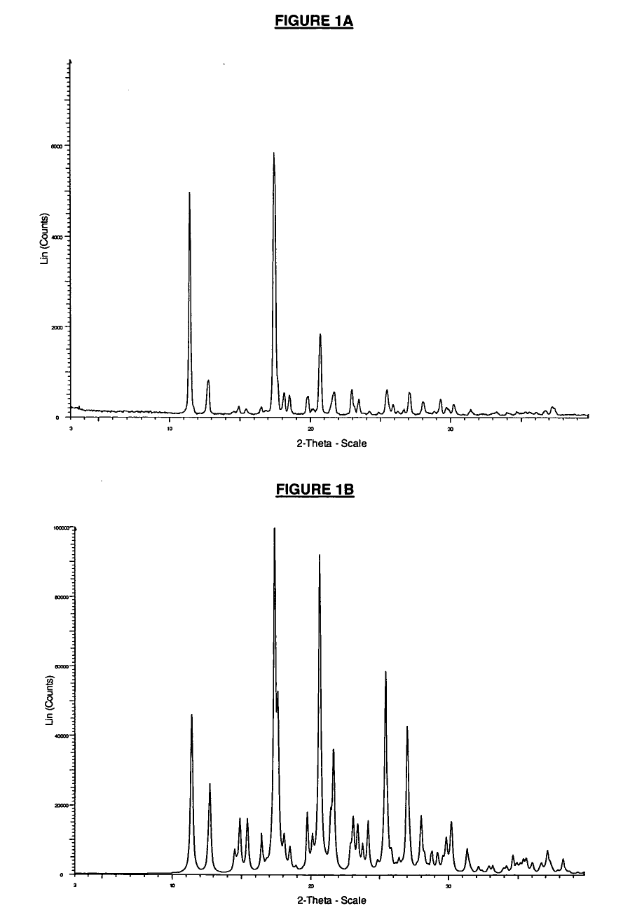

Form A is more stable than Form B and is thus the preferred polymorph for use in pharmaceutical compositions. Form A has crystalline habits that are birefringent rod-shaped and/or plate-shaped. Form A has a melt onset temperature of about 126° C., and is substantially fully crystalline by powder X-ray diffraction (PXRD), the pattern for which is shown in FIG. 1. As ascertainable from FIG. 1, Form A is characterized by a PXRD pattern having peaks at 2-θ values of approximately 11.4, 12.8, 17.4, 18.1, 18.5, 19.8, 20.7, 21.6, 23.0, 25.5 and 27.1. In one embodiment of the invention, the compound of formula I consisting essentially of polymorph Form A is contemplated.

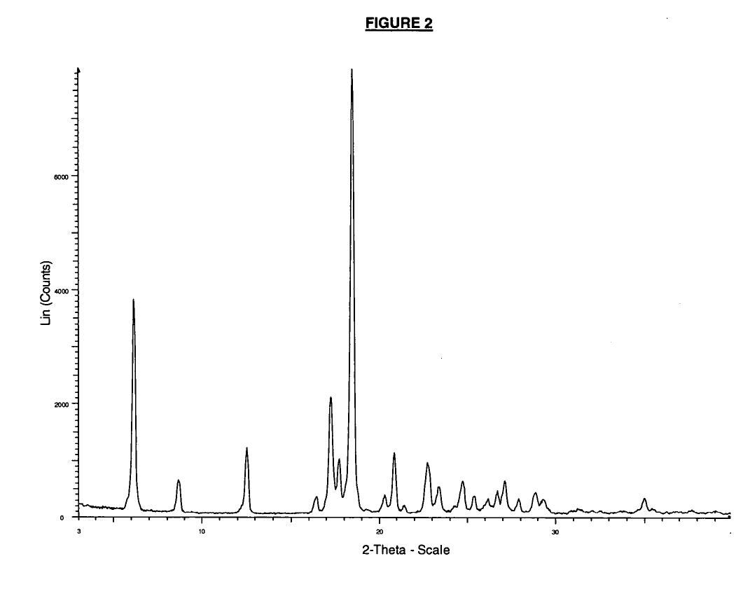

Form B is less stable than Form A. Form B has crystalline habits that are birefringent needles. Form B has a melt onset temperature of about 121° C. and is substantially fully crystalline by (PXRD), the pattern for which is shown in FIG. 2. As ascertainable from FIG. 2, Form B is characterized by a PXRD pattern having peaks at 2-θ values of approximately 6.1, 8.6, 12.5, 17.2, 17.7, 18.5, 20.9, 22.8, 23.4, 24.7 and 27.1. In another embodiment of the invention, the compound of Formula I consisting essentially of polymorph Form B is contemplated.

As appreciated by those of skill in the art, the scope of the invention includes isotopically-labeled compounds which are identical to Formula I save for the fact that one or more atoms are replaced by an atom having an atomic mass or mass number different from the atomic mass or mass number usually found in nature. Examples of isotopes that can be included into the compound of Formula I as contemplated by the invention include without limitation: isotopes of hydrogen, carbon, nitrogen and oxygen, such as for example, ²H, ³H, ¹³C, ¹⁴C, ¹⁵N, ¹⁸O. and ¹⁷O. Compounds of the present invention and pharmaceutically acceptable salts of same which contain the aforementioned isotopes and/or isotopes of other atoms are considered to be within the ambit of the invention. Certain isotopically-labeled compounds of the present invention, for example those into which radioactive isotopes such as ³H and ¹⁴C are incorporated are useful in drug and/or substrate tissue distribution assays. Tritiated, i.e. ³H, and carbon-14, i.e. ¹⁴C, isotopes are particularly preferred for their ease of preparation and detectability. Further, substitution with heavier isotopes such as deuterium, i.e. 2H, can afford certain therapeutic advantages resulting from greater metabolic stability, for example, increase in vivo half-life or reduced dosage requirements and hence may be preferred in some instances. Isotopically labeled compounds of this invention can generally be prepared by carrying out the procedures disclosed in the description and examples hereunder, with substitution of a readily available isotopically labeled reagent in lieu of a non-isotopically labeled reagent, using methods known in the art. Accordingly, reference to the compound of Formula I for use in the therapeutic methods and pharmaceutical compositions described herein also encompass isotopically-labeled forms of the same.

In another aspect, the present invention relates to a pharmaceutical composition useful in treating inflammation and/or pain subjects suffering from same, including without limitation mammals such as humans, livestock and companion animals such as dogs and cats. In particular, the pharmaceutical composition comprises a crystalline form of the compound 2-(3-difluromethyl-5-phenyl-pyrazol-1-yl)-5-methanesulfonyl-pyridine shown in Formula I, including e.g. Form A and Form B, as further characterized herein in an amount that is therapeutically effective amount to treat pain or inflammation, and a pharmaceutically acceptable carrier as conventionally known in the art. In a preferred practice the crystalline form is Form A. Without limitation, examples of serviceable carriers include diluents, fillers, water, various organic solvents and the like. The pharmaceutical composition may further include additives such as flavorings, binders, excipients and the like as routinely employed in the art.

In yet another aspect, the invention relates to a method of treating pain and inflammation comprising administering to a subject in need of such treatment therapeutically effective amount of a crystalline form of the compound 2-(3-difluromethyl-5-phenyl-pyrazol-1-yl)-5-methanesulfonyl-pyridine shown in Formula I, including Form A and Form B, as further characterized herein. Preferred subjects include mammals as aforesaid. The preferred crystalline form is Form A. Generally, administration can be effected by any method that enables delivery to the site of action. These methods include without limitation oral administration in the form of tablets or capsules and the like; intraduodenal routes; parenteral injection (including intravenous, subcutaneous, intramuscular, intravascular and infusion); topical and rectal. Oral administration is preferred, especially in the form of tablets or capsules.

Amounts that are therapeutically effective for treatment depend among other things on the subject species, the severity of the disorder or condition, the rate of administration and other factors as will be appreciated by those in the art. Without limitation, a therapeutically effective amount for dosage is between about 2 to about 6 mg per kg body weight per day, preferably about 3 to about 5 mg per kg body weight per day, more preferably about 4 mg per kg per day. As understood by those in the art, in some instances dosage levels below the lower limit of the aforesaid range will be adequate whereas in other instance dosages higher than the upper limit recited above can be employed without incurring undue or harmful side effects. Preferably, for larger dosages especially, the dosage form is divisible into several smaller doses to be administered throughout the day. As will be appreciated by those in the art, other anti-inflammatory, analgesics or even other medicaments can be employed in conjunction with the crystalline forms of the present invention for purposes of broader ranging therapy as may be desired.

The following examples are illustrative only. They are not constrictive on scope or preclusive of variations of the invention that are not otherwise expressly identified herein.

### EXAMPLES

Procedures:

Powder X-ray Diffraction analysis

The experimental conditions under which the powder X-ray diffraction was conducted are as follows: Cu anode; wavelength 1: 1.54056 angstrom; wavelength 2: 1.54439 angstrom (Relative Intensity: 0.500); range #1- coupled: 3.000 to 40.000; step size: 0.040; step time: 1.00; smoothing width: 0.300; and threshold: 1.0.

Single crystal X-ray analysis

Data collection: Bruker CCD diffractometer, Cu anode: wavelength 1.54178 angstrom; room temeprature;

Data analysis: Atomic scattering factors were taken the International Tables for X-ray Crystallography (Vol. IV, pp. 55, 99, 149 Birmingham: Kynoch Press, 1974). All crystallographic calculations were facilitated by the SHELXTL system G. M. Sheldrick, SHELXTL, User Manual, Nicholet Instrument Co., 1981). A trial structure was obtained by direct methods.

Calculation of PXRD pattern from single crystal data

The single crystal structural data provide the cell dimensions, space group and atomic positions of a crystal form. These parameters are used as the basis to calculate a perfect powder pattern of that crystal form. Comparing the calculated PXRD pattern and the experimental pattern will confirm whether a powder sample corresponds to an assigned single crystal structure. The results are displayed in the overlaid powder X-ray diffraction patterns with the lower pattern as the calculated from single crystal data and the upper one as a representative experimental pattern. A match between the two patterns indicates the agreement between powder sample and the corresponding single crystal structure.

### Example 1

### Preparation of 2-(3-difluromethyl-5-phenyl-pyrazol-1-yl)-5-methanesulfonyl-pyridine

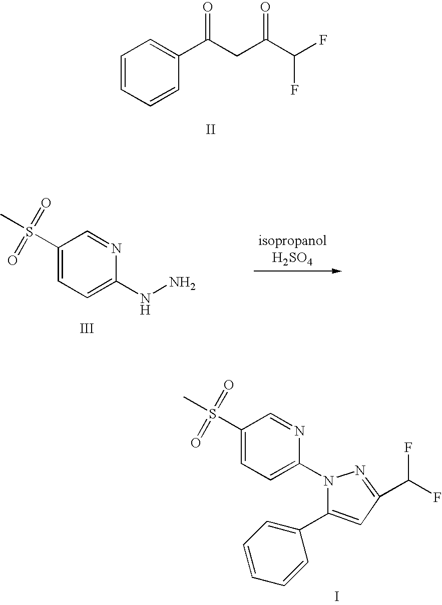

5 g of compound II was dissolved in 150 ml of isopropanol (IPA). To this solution was added 5 g of compound III and about 1.5 ml of concentrated H₂SO₄. The resultant solution was refluxed for 3 hr. after which it was cooled to room temperature in an ice bath. Then 200 ml of H₂O was added. The solution clouded and a white precipitate was formed. The solids were granulated for 3 hr., then filtered on a buchner funnel giving 7.43 g of off-white solid, which was dried overnight to provide 2-(3-difluromethyl-5-phenyl-pyrazol-1-yl)-5-methanesulfonyl-pyridine, Formula I (84% yield).

### Example 2

### Preparation of 2-(3-difluromethyl-5-phenyl-pyrazol-1-yl)-5-methanesulfonyl-pyridine

### Form A

1.0 g of the 2-(3-difluromethyl-5-phenyl-pyrazol-1-yl)-5-methanesulfonyl-pyridine prepared in accordance with Example 1 was dissolved in 15 ml of ethanol (EtOH) and heated to reflux. The resulting solution was immediately cooled to ambient temperature whereupon a white precipitate was formed. Stirring occurred for 3 hrs. after which the solution was filtered giving 830 mg of fine white powder This product was characterized by PXRD (Table 1) and DSC. PXRD revealed it to be crystalline, with the most intense peaks representatively observed at 2-θ values of approximately 11.4, 12.8, 17.4, 18.1, 18.5, 19.8, 20.7, 21.6, 23.0, 25.5 and 27.1, as shown in FIG. 1A. DSC revealed the crystals of Form A had a melt onset of about 125.5° C. with a heat of fusion of about 92 J/g. The crystalline habits of Form A were determined to be rod-shaped and plate-shaped. Calculated PXRD is at FIG. 1B.

TABLE 1
List of PXRD peaks for Form A (2 theta ± 0.1°)

| | | Calc. Form A | |
|---|---|---|---|
| Experimental Form A | | | Rel. |
| Angle 2-Theta ° | Rel. Intensity % | Angle 2-Theta ° | Intensity %* |
| 11.4 | 84.9 | 11.4 | 45.9 |
| 12.8 | 13.8 | 12.7 | 26.1 |
| 14.6 | 2.0 | 14.5 | 7.2 |
| 14.9 | 3.9 | 14.9 | 16.2 |
| 15.4 | 2.7 | 15.4 | 16.2 |
| 16.5 | 3.6 | 16.4 | 11.9 |
| 17.4 | 100.0 | 17.4 | 100.0 |
| | | 17.6 | 52.8 |
| 18.1 | 9.1 | 18.0 | 11.9 |
| 18.5 | 8.1 | 18.5 | 8.1 |
| 19.8 | 7.8 | 19.8 | 18.0 |
| 20.2 | 3.1 | 20.2 | 11.9 |
| 20.7 | 31.3 | 20.6 | 92.2 |
| | | 21.4 | 19.4 |
| 21.6 | 9.5 | 21.6 | 36.0 |
| 23.0 | 10.3 | 22.9 | 9.2 |
| | | 23.1 | 16.8 |
| 23.4 | 6.6 | 23.4 | 14.6 |
| | | 23.8 | 9.1 |
| 24.2 | 2.0 | 24.2 | 15.6 |
| 25.5 | 10.2 | 25.4 | 58.5 |
| 25.9 | 4.7 | 25.9 | 7.9 |
| 26.3 | 2.0 | 26.4 | 5.0 |
| 27.1 | 9.2 | 27.0 | 42.7 |
| 28.1 | 5.8 | 28.0 | 17.0 |
| 28.8 | 2.0 | 28.8 | 6.8 |
| 29.3 | 6.6 | 29.2 | 6.4 |
| 29.7 | 3.5 | 29.6 | 5.6 |
| | | 29.8 | 10.8 |
| 30.3 | 4.6 | 30.2 | 15.4 |
| 31.5 | 2.8 | 31.3 | 7.6 |
| 34.7 | 2.1 | 34.6 | 5.6 |
| 37.2 | 3.9 | 37.1 | 7.0 |

*The relative intensity may change with particle size and shape
*Missing peaks in experimental PXRD are due to either unresolved (±0.2°) or low intensity

### Example 3

### Preparation of 2-(3-difluromethyl-5-phenyl-pyrazol-1-yl)-5-methanesulfonyl-pyridine

### Form B

1.0 g of the 2-(3-difluromethyl-5-phenyl-pyrazol-1-yl)-5-methanesulfonyl-pyridine prepared in accordance with Example 1 was dissolved in 15 ml of IPA and heated to reflux. The resulting solution was immediately cooled to ambient temperature whereupon a white precipitate was formed. The solution thickened and 15 ml of IPA was added as a diluent. Stirring occurred for 3 hrs. after which the solution was filtered giving 820 mg of feathery white solid. This product was characterized by PXRD (Table 2) and DSC. PXRD revealed it to be crystalline, with the most intense peaks representatively observed at 2-θ values of approximately 6.1, 8.6, 12.5, 17.2, 17.7, 18.5, 20.9, 22.8, 23.4, 24.7 and 27.1, as shown in FIG. 2. DSC revealed the crystals of Form B had a melt onset of about 121.6° C. with a heat of fusion of about 78 J/g. The crystalline habits of Form B were determined to be needle shaped.

TABLE 2
List of PXRD peaks for Form B (2 theta ± 0.1°)

| Experimental Form B | | |
|---|---|---|
| | Angle 2-Theta ° | Intensity %* |
| | 3.5 | 2.6 |
| | 6.1 | 48.5 |
| | 8.6 | 8.2 |
| | 12.5 | 15.5 |
| | 16.4 | 4.5 |
| | 17.2 | 26.8 |
| | 17.7 | 13.0 |
| | 18.5 | 100.0 |
| | 20.3 | 4.9 |
| | 20.9 | 14.4 |
| | 21.4 | 2.6 |
| | 22.8 | 12.2 |
| | 23.4 | 6.7 |
| | 24.3 | 2.5 |
| | 24.7 | 8.0 |
| | 25.4 | 4.6 |
| | 26.2 | 4.1 |
| | 26.7 | 5.9 |
| | 27.1 | 8.1 |
| | 27.9 | 4.1 |
| | 28.9 | 5.5 |
| | 29.3 | 3.9 |
| | 35.0 | 4.2 |

*The relative intensity may change with particle size and shape

What is claimed is:

1. A crystalline form of 2-(3-difluromethyl-5-phenyl-pyrazol-1-yl)-5-methanesulfonyl-pyridine.

2. The crystalline form of the 2-(3-difluromethyl-5-phenyl-pyrazol-1-yl)-5-methanesulfonyl-pyridine of claim 1 wherein said crystalline form is selected from the group consisting of:
a) Form A characterized by a powder X-ray diffraction pattern having peaks at 2-θ values of approximately 11.4, 12.8, 17.4, 18.1, 18.5, 19.8, 20.7, 21.6, 23.0, 25.5 and 27.1; and
(b) Form B characterized by a powder X-ray diffraction pattern having peaks at 2-θ values of approximately 6.1, 8.6, 12.5, 17.2, 17.7, 18.5, 20.9, 22.8, 23.4, 24.7 and 27.1.

3. The crystalline form of claim 2 wherein Form A has a melt onset temperature of about 126° C.

4. The crystalline form of claim 2 wherein Form B has a melt onset temperature of about 121 ° C.

5. A pharmaceutical composition comprising the crystalline form of 2-(3-difluromethyl-5-phenyl-pyrazol-1-yl)-5-methanesulfonyl-pyridine according to claim 1 in an amount therapeutically effective to treat pain or inflammation; and a pharmaceutically acceptable carrier.

6. The pharmaceutical composition of claim 5 wherein said crystalline form is Form A.

7. The pharmaceutical composition of claim 5 wherein said crystalline form is Form B.

8. A method of treating pain and inflammation comprising administering to a subject in need of such treatment a therapeutically effective amount of the crystalline form of 2-(3-difluromethyl-5-phenyl-pyrazol-1-yl)-5-methanesulfonyl-pyridine as claimed in claim 1.

9. The method of claim 8 wherein said crystalline form is Form A.

10. The method of claim 8 wherein said crystalline form is Form B.

11. A method of making crystalline Form A of 2-(3-difluromethyl-5-phenyl -pyrazol-1-yl)-5-methanesulfonyl-pyridine comprising:
dissolving 2-(3-difluromethyl-5-phenyl-pyrazol-1-yl)-5-methanesulfonyl-pyridine in ethanol under conditions effective to form a precipitate; and
collecting said precipitate which is crystalline Form A of 2-(3-difluromethyl-5-phenyl -pyrazol-1-yl)-5-methanesulfonyl-pyridine.

12. The method of claim 11 wherein said conditions effective to form said precipitate include heating and agitation.

13. A method of making crystalline Form B of 2-(3-difluromethyl-5-phenyl -pyrazol-1-yl)-5-methanesulfonyl-pyridine comprising:
dissolving 2-(3-difluromethyl-5-phenyl-pyrazol-1-yl)-5-methanesulfonyl-pyridine in isopropanol under conditions effective to form a precipitate; and
collecting said precipitate which is crystalline Form B of 2-(3-difluromethyl-5-phenyl -pyrazol-1-yl)-5-methanesulfonyl-pyridine.

14. The method of claim 13 wherein said conditions effective to form said precipitate include heating and agitation.

15. The crystalline Form A of 2-(3-difluromethyl-5-phenyl-pyrazol-1-yl)-5-methanesulfonyl-pyridine characterized by a powder X-ray diffraction pattern having peaks at 2-θ values of approximately 11.4, 12.8, 17.4, 18.1, 18.5, 19.8, 20.7, 21.6, 23.0, 25.5 and 27.1; an onset melt temperature of about 126° C.

16. The crystalline Form B of 2-(3-difluromethyl-5-phenyl-pyrazol-1-yl)-5-methanesulfonyl-pyridine characterized by a powder X-ray diffraction pattern having peaks at 2-θ values of approximately 6.1, 8.6, 12.5, 17.2, 17.7, 18.5, 20.9, 22.8, 23.4, 24.7 and 27.1; an onset melt temperature of about 121° C.
